(19) United States
(12) Patent Application Publication
(10) Pub. No.: US 2019/0350566 A1
(43) Pub. Date: Nov. 21, 2019

(54) DEVICE FOR THE ASEPTIC COLLECTION OF URINE

(71) Applicant: Georgeta Maria COSTA ALVES DE OLIVEIRA, Vila Nova de Gaia (PT)

(72) Inventor: Georgeta Maria COSTA ALVES DE OLIVEIRA, Vila Nova de Gaia (PT)

(21) Appl. No.: 16/479,363

(22) PCT Filed: Jan. 31, 2017
(86) PCT No.: PCT/PT2017/000005

§ 371 Date: Jul. 19, 2019

(51) Int. Cl. A61B 10/00

(52) U.S. Cl. CPC A61B 10/007; A61B 2503/06

(57) ABSTRACT

A device for the aseptic collection of urine for subjects who do not have bladder 5 control, which comprises a housing made of water-soluble and absorbent material of flattened cylindrical shape provided with an adhesive zone on its outer surface, and a urine collecting container comprising a bag of flexible material, the base of which has an edge zone with 10 passage holes and a central zone, wherein said bag is made of flexible material and its edge zone is made of an elastic and semi-rigid material, which, in its initial position, is folded, overlaid on itself under tension, and said storage zone, in said initial position, is folded 15 over itself, said bag of flexible material being inserted into said housing of water-soluble and absorbent material.

### TECHNICAL FIELD OF THE INVENTION

The present invention relates to the field of medical devices and consists of a device which allows the aseptic collection of urine by a non-invasive method in subjects having no bladder control.

### OBJECT OF THE INVENTION

The present invention is directed to a device for the aseptic collection of aseptic urine to be used by subjects having no bladder control, especially children, by means of which aseptic urine can be obtained, which is crucial for the diagnosis of urinary infections, i.e., a device that does not give rise to false positives.

### INVENTION BACKGROUND

For subjects having no bladder control, especially children, the only available aseptic urine collection devices are the catheter and the puncture device (needle and syringe), whose methods of application, namely catheterization and suprapubic puncture, are invasive/traumatic and rely on the action of a health professional.

There are also other devices for urine collection widely used in children with no bladder control, consisting of adhesive collection bags, which are plastid bags with an opening to be placed around the urogenital area with the aid of a strip of adhesive. Material, and which allow for the urine to be collected while the child urinates. These collection devices are associated with a high rate of false positives during the detection of urinary tract infections, since contamination of the urine by fecal bacteria or urethral colonization bacteria usually occurs, i.e., they do not allow aseptic collection of urine.

Examples of such devices include those described in GB1051875, WO0000111, SE333792, U.S. Pat Nos. 3,406,690, 3,523,537, EP1089682 and U.S. Pat. No. 3,200,415.

Other devices intended to obviate the problem of contamination include those of U.S. Pat. Nos. 4,557,274, 4,492,258, 3,881,465 and EP1560522.

EP1560522 discloses a device for the aseptic collection of urine, which is regarded as the closest prior art of the present invention, comprising a urine collection bag with an absorbent material around an opening located in the upper side of the bag, to be placed in the urethral region. The urine reaches its opening after passing through a channel initially closed by a delayed opening shutter consisting of a soluble adhesive material which absorbs the urine along this channel and dissolves, so that the final urine reaches the urine collection bag through the open channel. This device, however, does not guarantee urine asepsis due to the high probability of the contaminated urine (first urine absorbed by the soluble material) contacting with the subsequent urine.

### DESCRIPTION OF THE INVENTION

The aseptic urine collection device according to the present invention comprises a water-soluble absorbent material housing and a collecting vessel. This housing prevents the contaminated urine (first urine absorbed by the soluble material) from contacting with the subsequent urine and solves the problem of urine contamination by the bacteria present in the urogenital area, by eliminating the first (contaminated) urine stream from the final collection, as described next, when the mode of operation of said device will be explained.

The water-soluble housing, within which the said collecting container is housed in the folded state, has a substantially cylindrical tubular shape. The water-soluble housing has on its exterior an adhesive zone in an ergonomic shape so making it suitable to be, adhered to the patient's pubic area.

The collecting container comprises a collection bag made of a flexible material film, preferably plastic, which may have any ergonomic shape suitable for the intended purpose, preferably a frustoconical shape, and is provided with a base on the periphery of which an edge zone is defined made of a semi-rigid material but having sufficient elasticity to be suitably folded so as to enable the collection bag to be introduced into said water-soluble housing in a folded condition under tension, being maintained in said condition by said housing. A central zone, which can be closed or opened, is delimitated in said base by said edge zone.

When said central zone is closed, said edge zone provided on the periphery of said base of said bag presents external passage holes that allow the urine to be collected to enter. When said central zone is open, the opening may have a film which can act as a shutter for the collected urine, but allows the urine to enter easily into said collection bag.

The device according to the present invention is intended to be applied in a diaper to be placed on the patient, and to be held against the patient's pubic area by the adhesion to the pubic area of said adhesive zone provided on the outside of said water-soluble housing and by said diaper.

The urine collecting container, according to the present invention, may be made from a unitary piece or one or more sealed pieces.

The device according to the present invention is sterilizable and should be packaged in a package for sterilization.

The device according to the present invention is disposable, i.e., it is intended for single use only.

The device according to the present invention operates as follows:
the device is made to adhere to the properly disinfected pubic area and then is properly placed in a diaper, which will assist in maintaining the device against the patient urogenital area;
upon initiation of urination, the first urine stream will be absorbed by the water-soluble housing and will dissolve the water-soluble housing;
the housing is then torn with the aid of unfolding of said edge zone of said collection bag, and the housing rupture zones contaminated with the first stream of urine are removed from the urogenital area, preventing contamination of the following urine stream, the edge zone of the bag opening being properly positioned in said urogenital area;
after removal of the diaper and the device, said collection bag containing urine is shut and the collected sample is sent for analysis, or the collected sample is withdrawn from said collection bag by means of a suitable device and sent for analysis.

### BRIEF DESCRIPTION OF THE DRAWINGS

For a more complete understanding of the present invention, reference is now made to the following description of a presently preferred embodiment thereof, in conjunction with the accompanying drawings, in which:

FIG. 1 is a schematic view of the present invention device;

FIG. 2 is a schematic view of the water-soluble tubular housing;

FIG. 3 is a view of the urine collecting container in the open condition.

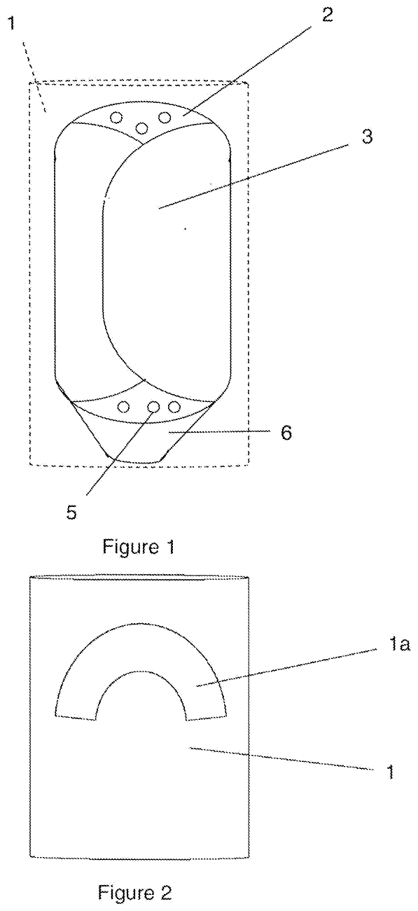

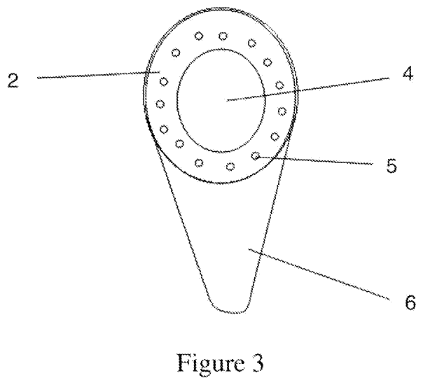

The device shown in FIG. 1 comprises a water-soluble absorbent material housing 1, which surrounds the collecting container 2.

In FIG. 1, the water-soluble absorbent material housing 1 is shown by dashed lines, with the urine collecting container 2 in the folded and tensioned state therein being transparently visible. In this figure, the collection bag 3 is partially visible and the collar attached to the periphery of the opening of said collection bag 3 is also partially visible.

The water-soluble and absorbent material housing 1, depicted in FIG. 2, has a flattened cylindrical tubular shape and is provided centrally on its surface with a strip of adhesive material 1a, in the shape of a circular half-crown, which is to be made to adhere to the pubic region of the patient.

The urine collecting container comprises a frustoconical shaped collection bag 3 made of a plastic film, the base of which has throughout its periphery an edge zone 2 made of a semi-rigid material with sufficient elasticity to be folded and inserted into the tubular housing 1 and return to its original shape when the tubular housing 1 is torn by the joint action of its dissolution due to contact with the first stream of urine and the elastic deployment of said annular edge 2.

The central zone 4, initially covered by the overlapping edge zone 2 and the water-soluble absorbent material housing 1, opens when this housing dissolves in contact with the first stream of urine, as indicated above, allowing the collection of the subsequent urine, which drains through one or more holes 5 to a contiguous storage zone 6.

The urine collection bag 3 may have various shapes and sizes, depending on its use, i.e., whether it is to be used on infants (0-3 years), or on older children and incontinent adults, female or male. The preferred form is concave oval taking into account the anatomy of the urogenital region, with the approximate dimensions of this area.

The volume of the storage zone 6 of the urine collection bag 3 varies according to the age of the child/adult, taking also into account the mean volume of urine per micturition.

It will be understood that the above embodiment has been provided by way of example only and is not limitative of the scope of the invention.

1-7. (canceled)

8. A device for the aseptic collection of urine for subjects who do not have bladder control, which comprises:
a housing and a urine collecting container comprising a bag of flexible material, the base of which has an edge zone with passage holes and a central zone, wherein it comprises a housing and said housing is made of water-soluble and absorbent material of flattened cylindrical shape provided with an adhesive zone on its outer surf ace and the edge zone of said bag is made of an elastic and semi-rigid material, which, in its initial position, is folded, overlaid on itself under tension, and the storage zone of said bag, in said initial position, is folded over itself, said bag of flexible material being inserted into said housing of water-soluble and absorbent material.

9. The device according to claim 8, wherein, when said central zone is open, the opening may have a film which can act as a shutter for the collected urine, but allows the urine to enter easily into said urine collection bag.

10. The device according to claim 8, wherein the flexible material of bag is a plastic material.

11. The device according to claim 8, wherein the elastic and semi-rigid material of said edge zone is a plastic material.

12. The device according to claim 8, wherein the urine collection bag is made from a unitary piece or one or more sealed pieces.

13. The device according to claim 8, wherein it is sterile and is packed in a sterile package.

14. The device according to claim 8, wherein it is of single use.
